(19) United States
(12) Patent Application Publication
(10) Pub. No.: US 2016/0090631 A1
(43) Pub. Date: Mar. 31, 2016

(54) METHOD FOR DETECTION OF FETAL ABNORMALITIES

(71) Applicant: James R. STELLING, Blue Point, NY (US)

(72) Inventor: James R. STELLING, Blue Point, NY (US)

(21) Appl. No.: 14/965,212

(22) Filed: Dec. 10, 2015

Related U.S. Application Data
(63) Continuation of application No. PCT/US2014/041947, filed on Jun. 11, 2014.
(60) Provisional application No. 61/833,653, filed on Jun. 11, 2013.

(51) Int. Cl. C12Q 1/68

(52) U.S. Cl. CPC C12Q 1/6883; C12Q 2600/158; C12Q 2600/156

(57) ABSTRACT

Disclosed are methods for non-invasive fetal genetic analysis involving enrichment of trophoblast cells in a maternal cervical sample, followed by isolation and genetic analysis of the isolated trophoblasts.

### CROSS-REFERENCE TO RELATED APPLICATIONS

This application claims priority to U.S. provisional application 61/833,653, filed Jun. 11, 2013, which is incorporated herein in its entirety.

### BACKGROUND OF THE DISCLOSURE

Genetic diseases and chromosomal disorders are unfortunately encountered commonly during pregnancies of many women. The rate of chromosomal abnormalities, such as Down Syndrome (Trisomy 21) increase with advancing maternal age. These abnormalities often lead to very difficult medical conditions and decisions concerning these abnormalities. Current obstetrical care involves several methods to help women predict the likelihood of chromosomal abnormalities during pregnancy. These techniques include some noninvasive tools such as ultrasound to measure nuchal thickness, as well as blood work such as the quad screen or triple screen. These noninvasive tests combined with patient's age will help predict the rate of having a chromosomal abnormal pregnancy. These tests will then help decide whether to proceed with more invasive testing that will more firmly determine fetal status. These tests include a chorionic villus sampling performed at approximately 10-12 weeks of pregnancy or an amniocentesis traditionally done at 16-20 weeks.

Unfortunately, while these tests are highly accurate they do carry significant risk. The estimated rate of a pregnancy loss after an amniocentesis is approximately 1/300. Therefore these invasive tests are usually limited to those patients considered to be at highest risk, based upon age and the mentioned biochemical and ultrasound markers. Because of the risk and fear of these procedures many patients do not elect to undergo these procedures, potentially leading to the birth of more affected children. Even if patients do not elect to have a termination of an affected pregnancy knowing the pregnancy is affected could help mentally and medically prepare a patient to have an affected child. A noninvasive and more accurate test than currently available would help many of these patients. In theory if the test was accurate, reasonable cost, and posed no/minimal risk all pregnant women could be offered this prenatal testing.

This desire for this test has led many investigators to try to develop this technology. Most of the current research focus is based upon finding fetal cells or fetal DNA in the blood of the pregnant women. This direction has been hampered by the low percentage of fetal cells found in maternal blood. In addition when fetal cells have been found they have not always been from the fetus that the women is currently pregnant with, as fetal cells have been found from previous pregnancies. Cell free fetal DNA has also more recently been used but is less accurate then the invasive tests available. Another promising direction is trying to obtain fetal cells from the cervix of the pregnant woman. This has been done by doing uterine lavages, cotton swabs, and more recently by using the cytobrush that is currently used for routine Pap smears. The fetal cells are able to be separated from the maternal cells using different fetal trophoblastic antibodies along with a cell sorter. These fetal cells can then be genetically analyzed by a variety of techniques. The most common in the past and still being studied is FISH (Fluoresence In-situ Hybridization).

Improved methods of non-invasive fetal testing, with lower costs and reduced skill requirements, are desired.

### BRIEF SUMMARY OF THE DISCLOSURE

This disclosure provides methods of performing a non-invasive prenatal test to evaluate fetal chromosomal and single gene disorders using trophoblast cells obtained from the cervix of pregnant women.

Disclosed herein are methods of fetal genetic analysis, which include enriching fetal trophoblasts in a sample of cells isolated from the maternal cervix, isolating at least one fetal trophoblast from the sample enriched in trophoblasts, and performing genetic analysis on the at least one trophoblast.

In one embodiment, the fetal trophoblasts are enriched in the sample by density gradient centrifugation, flow cytometry, immunobeads, or collagen adhesion. In another embodiment, the at least one trophoblast is isolated from the enriched sample by flow cytometry, immunobeads, or micromanipulation. In a further embodiment, all steps of the method are performed without cell staining.

In another embodiment, the genetic analysis is performed utilizing genetic analysis as is frequently used in pre-implantation genetic diagnosis (PGD) of embryos. PGD has been optimized to work on very few cell numbers even as low as a single cell. Techniques used include FISH, array comparative genomic hybridization (aCGH), karyomapping with single polymorphisms (SNPs), and next generation sequencing (Next Gen or NG).

### BRIEF DESCRIPTION OF THE FIGURES

FIG. 1. Stained cervical cell sample. Fetal trophoblast is evident as small, spherical cell shape and large nucleus, relative to surrounding maternal cervical cells with larger, irregular shape and small nuclei.

FIG. 2. Unstained cervical cell sample. Trophoblast-like cells are small, spherical cells (circled) surrounded by larger cells with morphology of maternal cervical cells.

FIG. 3. Enrichment increases the proportion of trophoblast-like cells in a cell sample. Sample is stained for demonstration.

FIG. 4. Genetic analysis result representative of female without chromosomal abnormalities.

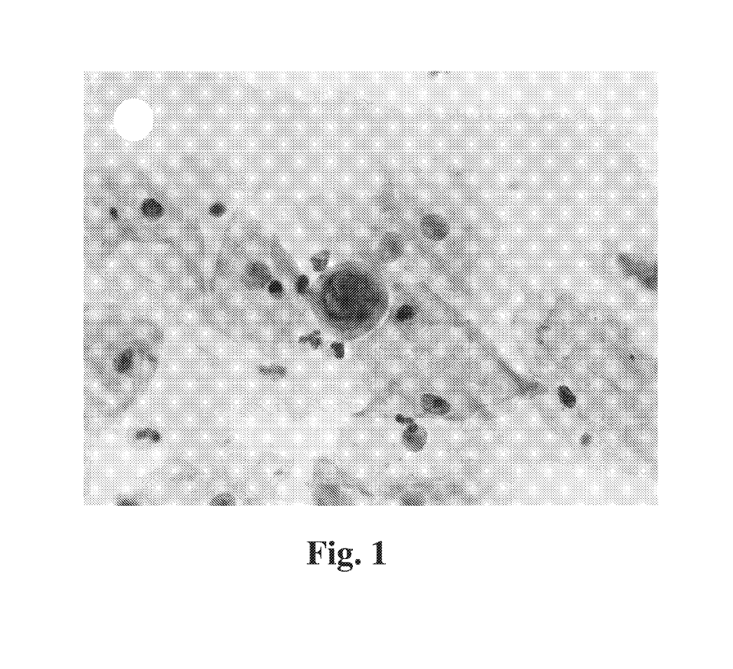

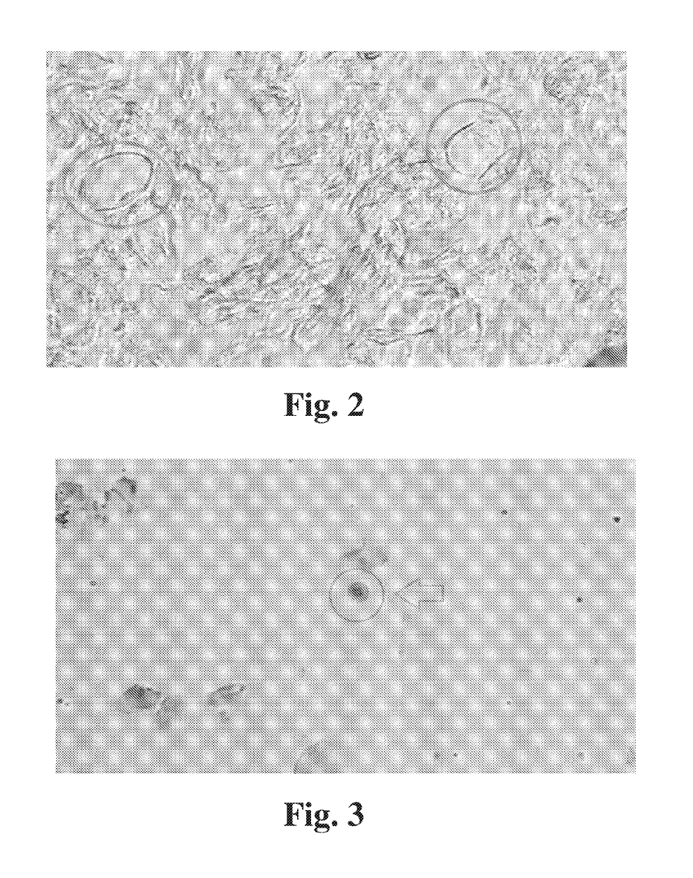

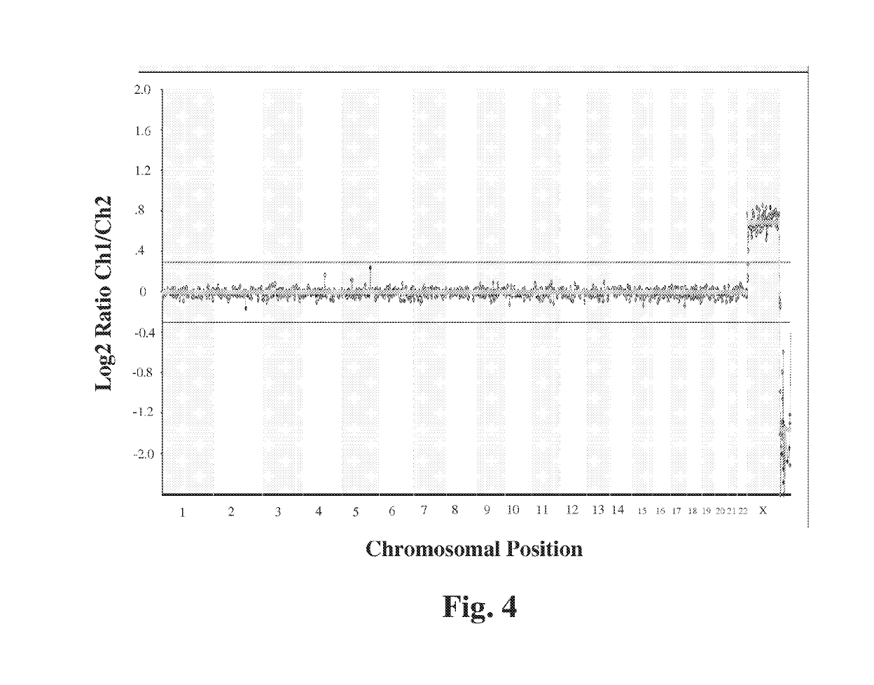

### DETAILED DESCRIPTION OF THE DISCLOSURE

This disclosure provides methods of evaluating fetal genetic disorders. The methods include enriching fetal trophoblasts in a sample of cells isolated from the maternal cervix, isolating at least one fetal trophoblast from the sample enriched in trophoblasts, and performing genetic analysis on the at least one trophoblast.

A trophoblast is an epithelial cell derived from the placenta of a mammalian embryo or fetus; trophoblasts typically contact the uterine wall. There are three types of trophoblast cells in the placental tissue: the villous cytotrophoblast, the syncytiotrophoblast, and the extravillous trophoblast, and as such, the term “trophoblast” as used herein encompasses any of these cells. The villous cytotrophoblast cells are specialized placental epithelial cells which differentiate, proliferate and invade the uterine wall to form the villi. Cytotrophoblasts, which are present in anchoring villi can fuse to form the syncytiotrophoblast layer or form columns of extravillous trophoblasts (Cohen S. et al., 2003. J. Pathol. 200: 47-52). Any of these cells can be shed and recovered from the maternal cervix. By “fetal cells” is meant cells derived or originating from tissue of a fetus or embryo. The terms “fetal cells” and “trophoblasts” are used interchangeably herein.

By “fetal-like cells” or “trophoblast-like cells” is meant cells that have morphology or characteristics associated with trophoblasts. A typical trophoblast measures between 10 and 30 microns and shows a high nuclear to cytoplasmic ratio, for example, a nucleus to cytoplasm ratio between 1:1 to 10:1. Typical trophoblast characteristics include expression of cell surface markers such as insulin-like growth factor (IGF)-II, NDOG-5, proliferating cell nuclear antigen (PCNA), human leucocyte antigen framework antigen (W6/32) and a distinct set of integrins including alpha 1, alpha 3, alpha 5, alpha v and beta 1 subunits and alpha v beta 3/beta 5 vitonectin receptor. Additional trophoblast characteristics include ability to adhere to and/or invade a collagen material.

The trophoblast-containing cell sample can be obtained from a pregnant woman beginning at the 5^{th} week of gestation. The sample and methods can also be utilized for genetic analysis of a product of conception or placental tissue sample.

A cell sample can be obtained from the cervix of a pregnant woman by any method known in the art. “Cell sample” and “cervical cell sample” are used interchangeably herein. Preferred methods involve cell removal from the cervix using a cell collection device selected from a gynecological swab with cotton, nylon, or plastic fiber-tipped end, a cervical brush (for example, PAPETTE Cervical Cell Collector, Wallach Surgical Devices), a gynecological spatula (for example, Wooden Ayre gynecological spatula, Adlin Medical), or a cytobrush (for example, disposable cervical brush, DiaPath Spa).

Following cell collection, the end of the cell collection device containing the cells is swirled or inserted into an appropriate cell culture media to transfer the cells into the media. Exemplary media for cell collection can be methanol based, such as PRESERVCYT solution used in the THINPREP Pap system (Hologics, Inc). Media based on 1× phosphate buffered saline can also be used. In one example, the cell collection device is notched so that the sample-collection end can be inserted into the media and the handle can be broken to immerse the sample-collection end in the media for transportation of the sample collection end within the media.

A typical cervical sample contains hundreds of thousands of cells, extensive cervical mucus, and a large amount of cellular debris. The number of fetal cells present in a typical unprocessed cervical cell sample is small, ranging anywhere from 1 in 1,000 to 1 in 10,000 cells or even fewer than 1 in 10,000 cells. In order to increase the proportion of fetal cells in a sample, the cell sample can be enriched in fetal cells. By “enriched” is meant the sample is processed or subjected to one or more treatments to increase the proportion of trophoblasts and trophoblast-like cells in the sample. The trophoblasts can be enriched by 10-fold, 50-fold, 100-fold, 500-fold, or 1,000-fold or more in the sample by using the methods provided herein.

Preferred methods for enriching trophoblasts in a sample of cervical cells include density gradient centrifugation, immunoaffinity methods such as flow cytometry and immunobeads, and collagen adhesion.

Density gradient centrifugation involves ultracentrifugation of substances in a concentrated solution which, at equilibrium, exhibits a concentration (hence density) gradient increasing in the direction of centrifugal force and the substances of interest collect in layers at the levels of their densities. In one embodiment, a silane-coated silica particle density gradient (ALLGRAD, LifeGlobal Media, Inc.) is used. The cell sample is centrifuged to collect cells, and the cell pellet iss isolated and placed in a prepared density gradient conical tube with 10-15 layer of varying dilutions of density gradient (ranging from 5%-70%). The tubes are centrifuged at 1200 g for 5-30, preferably 10-20 minutes. The fraction of sample between 30%-50% density gradient will contain trophoblasts.

Immunoaffinity methods include affixing an antibody to a physical carrier or fluorescent label. Sorting steps can then be used to positively or negatively enrich for the desired cell type after the antibody binds to its target present on the surface of the cells of interest. Such methods include affinity chromatography, particle magnetic separation, centrifugation, filtration, and flow cytometry (including fluorescence activated cell sorting; FACS).

In one embodiment, magnetic beads are used to enrich trophoblasts in a sample. Magnetic beads are known in the art, and are available commercially. Magnetic beads can be purchased that are coated with secondary specific binding members, for example secondary antibodies or streptavidin. Preferred magnetic beads of the present invention are from 0.02 to 20 microns in diameter, preferably from 0.05 to 10 microns in diameter, and more preferably from 0.05 to 5 microns in diameter, and even more preferably from 0.05 to 3 microns in diameter and are coated with either a secondary binding member such as streptavidin or a primary specific binding member such as an antibody that can bind a cell that is to removed from the sample. An example of a preferred magnetic bead is DYNABEAD (Life Technologies, Inc.). Where streptavidin coated beads are used, the primary specific binding member is preferably biotinylated (for example a biotinylated antibody) such that the streptavidin coated bead will bind a sample component that is bound to the biotinylated antibody through a streptavidin-biotin link. Methods of using magnetic beads in the capture of directly or indirectly bound cells are well known in the art.

Flow cytometry or a fluorescence activated cell sorter (“FACS”) detects and separates individual cells one-by-one from background cells.

Immunoaffinity can further enrich a sample by removing maternal cells, such as by binding and removing maternal cells with antibodies specific for maternal cell surface antigens, optionally followed by isolation of fetal cells, for example using trophoblast-specific antibodies, or density gradient centrifugation.

Antibodies directed against trophoblast specific antigens are known in the arts and include, for example, the HLA-G antibody, which is directed against part of the non-classical class I major histocompatibility complex (MHC) antigen specific to extravillous trophoblast cells (Loke, Y. W. et al., 1997. Tissue Antigens 50: 135-146), the anti human placental alkaline phosphatase (PLAP) antibody which is specific to the syncytiotrophoblast and/or cytotrophoblast (Lehner, K. et al., 2001, J. Histochemistry and Cytochemistry, 49: 1155-1164), the CHL1 (CD146) antibody which is directed against the melanoma cell adhesion molecule (MCAM) (Higuchi T., et al., 2003, Mol. Hum. Reprod. 9: 359-366), the CHL2 antibody which is directed against laeverin, a novel protein that belongs to membrane-bound gluzincin metallopeptidases and expressed on trophoblasts (Fujiwara H., et al., 2004, Biochem. Biophys. Res. 313: 962-968), the H315 antibody which interacts with a human trophoblast membrane glycoprotein present on the surface of fetal cells (Covone A E and Johnson P M, 1986, Hum. Genet. 72: 172-173), the FT1.41.1 antibody which is specific for syncytiotrophoblasts and the 103 antibody (Rodeck, C., et al., 1995. Prenat. Diag. 15: 933-942), the NDOG-1 antibody which is specific for syncytiotrophoblasts (Miller D., et al. Human Reproduction, 1999, 14: 521-531), the NDOG-5 antibody which is specific for extravillous cytotrophoblasts (Miller D., et al. 1999, Supra), the BC1 antibody (Bulmer, J. N. et al., Prenat. Diagn. 1995, 15: 1143-1153), the AB-154 or AB-340 antibodies which are specific to syncytio—and cytotrophoblasts or syncytiotrophoblasts, respectively (Durrant L et al., 1994, Prenat. Diagn. 14: 131-140), the protease activated receptor (PAR)-1 antibody which is specific for placental cells during the 7th and the 10th week of gestation (Cohen S. et al., 2003. J. Pathol. 200: 47-52), the glucose transporter protein (Glut)-12 antibody which is specific to syncytiotrophoblasts and extravillous trophoblasts during the 10th and 12th week of gestation (Gude N M et al., 2003. Placenta 24:566-570), the anti factor XIII antibody which is specific to the cytotrophoblastic shell (Asahina, T., et al., 2000. Placenta, 21: 388-393; Kappelmayer, J., et al., 1994. Placenta, 15: 613-623), the Mab FDO202N directed against the human placental lactogen hormone (hPLH) which is expressed by extravillous trophoblasts (Latham S E, et al., Prenat Diagn. 1996; 16(9):813-21).

In one embodiment, the trophoblasts are enriched from cervical cells by selecting for cells that adhere to collagen, such as by plating a sample on a collagen surface, or by use of collagen adhesion matrix (CAM). CAM is disclosed in U.S. Pat. No. 7,785,810, the contents of which are incorporated herein. CAM is provided, for example, in matrix coated tubes that allow for cell-specific isolation according to cell type. In another embodiment, cells are enriched for trophoblasts by culturing the cell sample on collagen-coated plates or wells, collagen I gels such as PURECOL (Sarstedt, Inc.), or MATRIGEL, for a period of 1 hour to 1 week, followed by washing the plates and removing adherent cells.

After enrichment, trophoblasts are isolated from the enriched sample. Preferred isolation methods include flow cytometry or immunobeads using the antibodies above, or micromanipulation. In micromanipulation, cells are visually inspected and “hand-picked” according to trophoblast morphological characteristics such as size (about 10-30 microns in diameter) high nuclear to cytoplasmic ratio (for example, a nucleus to cytoplasm ratio between 1:1 to 10:1).

Prior to genetic analysis, nucleic acids are preferably amplified. Nucleic acid is isolated from cells according to standard methodologies (Sambrook et al., Molecular Cloning, 2nd ed., Cold Spring Harbor Laboratory Press, CSH, 1.38-1.39, 1989). A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety. Other methods of amplification are ligase chain reaction (LCR), Qbeta Replicase, isothermal amplification, strand displacement amplification (SDA), PCR-like template- and enzyme-dependent synthesis using primers with a capture or detector moiety, transcription-based amplification systems (TAS), cyclical synthesis of single-stranded and double-stranded DNA, “RACE”, one-sided PCR, and di-oligonucleotide amplification.

Preferred methods for amplification of nucleic acids, including single cell whole genome amplification, are described in U.S. Pat. No. 8,206,913, the contents of which are incorporated by reference herein.

The phrase “genetic analysis” as used herein refers to any chromosomal, DNA and/or RNA-based analysis which can detect chromosomal, DNA and/or gene expression abnormalities, respectively in a cell of an individual (i.e., in the trophoblast cell of the present invention). Although exemplary methods of DNA analysis are known in the art, the disclosed methods can be used with any form of DNA analysis.

Different panels of genetic tests can be performed on the trophoblast nucleic acid, including, for example, analysis of chromosomal abnormalities, a standard panel for Fragile X, cystic fibrosis, spinal muscle atrophy, a “Jewish” panel, a panel as recommended by the American College of Medical Genetics, or a custom-designed panel based on the genetic background of the mother and/or father of the fetus.

### EXAMPLES

### Example 1

### Sample Collection

Cell sample from cervical swab. A Pap smear cytobrush is inserted through the external os to a maximum depth of 2 cm and removed while rotating it a full turn (i.e., 360°). In order to remove cells from the brush, the brush is swirled in a test tube or vial containing 2-3 ml of either a methanol-based solution such as THINPREP PRESERVCYT solution (Hologics, Inc.), or PBS with or without 10% serum albumin

### Example 2

### Trophoblast Enrichment by Density Gradient Centrifugation

Cells in medium were enriched by applying the sample to a silane-coated silica particle density gradient (ALLGRAD, LifeGlobal Media, Inc.) First the sample was centrifuged in collection media to concentrate cellular material and remove any methanol. The pellet was isolated and placed in a prepared density gradient conical tube with 10-15 layer of varying dilutions of density gradient (ranging from 5%-70%). The tubes were centrifuged at 1200 g for 10-20 minutes. The fraction of sample between 30%-50% density gradient was isolated, resuspended in 5 mL PBS with or without 10% serum albumin, and centrifuged at 1000 g for 5 minutes. The pellet was then isolated and resuspended in 1 mL PBS with 10% serum albumin.

Surprisingly, the inventors found that density gradient centrifugation could successfully enrich trophoblasts in a sample obtained from the maternal cervix. Enrichment of trophoblasts was unexpected because recovery of the few fetal cells present in a maternal cervical sample would not be anticipated using density gradient centrifugation, which is only used when there is assumed to be a sufficient number of cells in a sample to form a visible layer in the gradient. Here, the inventors found that density gradient centrifugation provided a fraction highly enriched in trophoblasts, which could then be isolated for further analysis.

### Example 3

### Trophoblast Enrichment by Collagen Adhesion

As an alternative to density gradient centrifugation, collagen adhesion is utilized to enrich trophoblasts in a sample. A cervical cell sample is seeded onto GROWCOAT collagen-1 coated plates (Sarstedt, Inc., Newton, N.C.) and cultured according to the manufacturer's instructions. Trophoblasts adhere to the collagen surface and cervical cells and debris can be removed by washing. Following the washing step, the trophoblast-enriched sample can be removed by enzymatic digestion, such as trypsin or HYQTASE (HyClone, Inc.) for further isolation of trophoblasts.

### Example 4

### Isolation from Enriched Sample by Micromanipulation

For isolation by micromanipulation, the enriched sample is plated in low wall dishes covered by mineral oil for tissue culture (Oil for Embryo Culture, Irvine Scientific). Dishes are prepared with a 20-50 footprinted trough of PBS with protein supplementation surrounded by two rows of 10-20 μL unsupplemented PBS droplets. The trough is scanned on an inverted microscope for potential fetal cells according to morphological characteristics such as size (about 10-30 microns in diameter) and nuclear to cytoplasmic ratio (for example, a nucleus to cytoplasm ratio of 1:1 to 10:1). Cells are then aspirated into a polished blastomere biopsy micro-pipete pulled to an inner diameter of 28-32 μm (Blastomere Biopsy Pipette, Origio) using a micromanipulator (Transferman NK2, Eppendorf USA.) The cell is then rinsed in the first PBS drop to decrease the risk of contamination with any free DNA or other cell type. The cell is then aspirated again and rinsed in the second PBS drop. At this point the cell is isolated and ready for genetic analysis.

### Example 5

### Single Cell Amplification

Amplification of single cell nucleic acid. Single cell nucleic acids are amplified prior to genetic analysis using the PICOPLEX WGA kit (Rubicon Genomics) for whole genome amplification, using the manufacturer's standard protocol for single cell genetic amplification. As a control, nucleic acids from maternal cells are also amplified by WGA or by other amplification methods.

### Example 6

### Genetic Analysis

After amplification, genetic analysis of the fetal and maternal nucleic acid is performed. Comparative genomic hybridization (aCGH), karyomapping with SNP (single nucleotide polymorphism) analysis and next generation sequencing methods are applied. Genetic analysis is used to identify possible DNA abnormalities, and to prove that the fetal cells' “DNA fingerprint” matches half of the maternal “DNA fingerprint”.

Fetal DNA is tested for aberrations according to current guidelines of the American College for Medical Genetics and/or the American Congress of Obstetricians and Gynecologists.

What is claimed is:

1. A method for fetal genetic analysis, comprising enriching fetal trophoblasts in a sample of cells isolated from the maternal cervix, isolating at least one trophoblast from the sample enriched in trophoblasts, and performing genetic analysis on said at least one trophoblast.

2. The method of claim 1, wherein the fetal trophoblasts are enriched in the sample by density gradient centrifugation, flow cytometry, immunobeads, or collagen adhesion.

3. The method of claim 1, wherein the at least one trophoblast is isolated from the enriched sample by flow cytometry, immunobeads, or micromanipulation.

4. The method of claim 1, wherein all steps of said method are performed without cell staining.

5. The method of claim 1, wherein single cell amplification is utilized to amplify the nucleic acid of said at least one trophoblast, prior to genetic analysis.

6. The method of claim 5, wherein said single cell amplification is whole genome amplification.

7. The method of claim 1, wherein the cell sample is isolated from the maternal cervix using a collection device selected from a swab, cervical brush, or cytobrush.

8. The method of claim 7, wherein the collection device is notched to facilitate breakage of the cell collection portion of said device into a collection medium.
